(19) United States
(12) Patent Application Publication
(10) Pub. No.: US 2004/0043039 A1
(43) Pub. Date: Mar. 4, 2004

(54) Method for inducing an immune response to polysaccharide bacterial antigens and to protein structures of virus capsides

(76) Inventors: Claude-Agnes Reynaud, Paris Cedex (FR); Jean-Claude Weill, Paris Cedex (FR)

Correspondence Address: OPPEDAHL AND LARSON LLP, P O BOX 5068, DILLON, CO, 80435-5068, US

(21) Appl. No.: 10/466,950

(22) PCT Filed: Jan. 22, 2002
(86) PCT No.: PCT/FR02/00240

(30) Foreign Application Priority Data
Jan. 22, 2001 (FR) 0100834

(51) Int. Cl. A61K 39/00; A61K 39/12

(52) U.S. Cl. 424/204.1; 514/54; 424/185.1

(57) ABSTRACT

The invention concerns an immunogenic composition, in a pharmaceutically acceptable carrier, for specifically stimulating a sub-population of B M⁺D⁺CD27⁺ cells so as to provide T-independent anti-bacterial response in said cells. The invention also concerns the use for producing T-independent anti-bacterial immune responses in subjects infected or susceptible of being infected by polysaccharide bacteria (streptococcus, meningococcus, pneumococcus, hemophilus influenza) or by protein capsid viruses (poliovirus, encephalomyocarditis virus, influenza virus).

### FIELD OF THE INVENTION

The present invention pertains to the field of immunization of warm-blooded animals, including man, against infectious agents. More specifically, it relates to means of triggering and/or reinforcing immune responses to infections and diseases caused by encapsulated bacteria with polysaccharide functions and/or by protein structures of virus capsids.

### TECHNOLOGICAL BACKGROUND OF THE INVENTION

The immune response to polysaccharide antigens develops gradually during the early years of life (H. Hidalgo et al., “Pre- and postimmunization antibody titers in children with recurrent infections,” Ann. All. Asthma. Immunol. 76 (1996), 341-346) and involves chiefly the IgG2s. Children under 2 years of age and elderly persons lack defenses against encapsulated bacteria with polysaccharide functions, whereas such individuals are able to respond to T-dependent (protein) antigens. This phenomenon, which has clinical implications, has not been completely elucidated to date.

Now, pneumococcus, meningococcus, Haemophilus influenzae and the streptococci are genera with polysaccharide capsule and functions varying according to the strain. By way of example, 84 different serotypes of Pneumococcus have been identified. Now this agent is the principal cause of bacterial pneumopathy, meningitis, sinusitis and otitis. Among these conditions, pneumococcic pneumonia accounts for nearly 5000 deaths per year in France, 90% of them in persons over 65 years of age, and 1 million deaths per year worldwide in children under 5 years (Lancet 354 [1999], editorial, 2011; F. Shann, “Pneumococcal vaccine: Time for another controlled trial,” Lancet 351 [1998], 1600-1601; G. R. Siber, “Pneumococcal disease: Prospects for a new generation of vaccines,” Science 265 [1994], 1385-1387).

Resistance to Pneumococcus antibiotics, formerly an exception, is becoming increasingly frequent, involving 20% to 35% of strains for the more commonly used antibiotics, the penicillins and the macrolides, thus bringing the problem of antipneumococcic and likewise antimeningococcic vaccination back into the foreground.

The early vaccines were aimed at the polysaccharide antigens of the virus capsules, and they elicited a T-independent B response. These vaccines, first 14-, then 23-valent, which were to cover 90% of the serotypes, had been presented as efficacious 20 years ago. It is now known (Shann 1998 supra) that they offer only partial protection against the septicemic forms, and even that they have shown no efficacy in the non-septicemic forms, which are of far more frequent occurrence; in particular, they have proved inactive in individuals under 2 years or over 50 years of age (A. Örtqvist et al., “Randomised trial of 23-valent pneumococcal capsular polysaccharide vaccine in prevention of pneumonia in middle-aged and elderly people,” Lancet 352 [1998], 399-403), and in the best of cases, their duration of action is brief. The result is inefficacy, or a very limited duration of efficacy, requiring numerous re-vaccinations in the case of traditional vaccines aimed against the polysaccharide antigens of the virus capsules, in individuals under 2 or over 50 years of age approximately.

For all these reasons, world vaccine policy remains confused. Whereas the health authorities of Italy, Spain and Belgium have developed programs of systematic vaccination, those of Germany, the Netherlands and France have disregarded the matter almost completely (Lancet 1999 supra, editorial), even though France is the principal European vaccine producer.

This situation of near failure in the prevention and treatment of infections, in particular pneumococcic and meningococcic infections in the young and the elderly, is however a major public health problem.

Here several strategies have seemed possible (Siber 1994 supra):

The conjugate vaccines, on the model of the anti-hemophilus B vaccine, the efficacy of which is clearly established, represent the 7 or 9 commonest serotypes, each coupled to an antigen protein, thus serving to obtain a much stronger immunological response by acting on the T-independent B lymphocytes. However, they present considerable problems of technology and production cost, for each polysaccharide antigen must be coupled selectively to the antigen protein in a suitable concentration, so that the vaccine thus constituted is really a mixture of several, in practice 7, different vaccines, very difficult to realize technically. Besides, with such vaccines other problems arise. Thus, the necessarily high dosage of protein antigen may induce severe reactions. Furthermore, there is a problem of antigen competition among the various subvaccines. But also, and above all, a major problem is that of the difficulty of choice of antigen proteins, diphtheria or tetanus toxins, or exterior envelope proteins of meningococcus (Siber 1994, supra; Shann 1998, supra).

An alternative solution consists in giving up the polysaccharide antigens and using the pneumococcic proteins or enzymes—pneumolysine, neuraminidase, aurolysine, hyaluronidase, surface A protein, surface A adhesine, etc. As yet, none of the immunization trials based on these choices has shown experimental evidence of efficacy (Siber 1994, supra; Shann 1998, supra).

A third solution under consideration would be to inject anti-CD40 antibodies simultaneously with conventional vaccines, possibly bringing about a polyclonal B activation (Shann 1998, supra).

However, none of these has provided a fully satisfactory answer to the health problem referred to above.

It has now been found, unexpectedly, that it is possible to induce and/or stimulate, or else negatively control, an immune response to the polysaccharide antigens and to the protein structures of virus capsids by specifically stimulating or inhibiting, as the case may be, a subpopulation of B cells, namely the sub-population B M⁺D⁺27⁺, including the cells of a B subpopulation that are not in a germinative center, advantageously in a targeted manner to produce a T-independent antibacterial response.

Means have thus been worked out to trigger and/or reinforce such an immune response in subjects having no immune response of their own, or whose immune response in question is inadequate—as is the case in infants under 2 years of age and in persons over age 65—as well as means of testing whether a suitable immune response has occurred, whether as a natural immune response or as a result of induction and/or stimulation of an immune response according to the present invention, that is to say, against bacterial polysaccharide antigens and protein structures of virus capsids.

The objects of the invention are a new vaccinal and immunogenic concept, a vaccine pertaining thereto, and a test protocol for diagnosis either of states calling for vaccination according to the invention or of the efficacy of a vaccination according to the invention in a subject who has been subjected thereto. Another object of the invention is the application of the said particular immunogenic concept to the preparation of means for inhibition of an autoimmune response.

### SUMMARY OF THE INVENTION

The means according to the invention for immunizing warm-blooded animals including man against infections and diseases caused by encapsulated bacteria having polysaccharide functions and/or by protein structures of virus capsids, comprise firstly an original vaccinal means, and secondly diagnostic means of evaluating states requiring vaccination according to the invention and/or the efficacy of a vaccination according to the invention on blood samples collected from a given subject.

### BRIEF DESCRIPTION OF THE DRAWINGS

The invention will now be illustrated in more detail, referring to the accompanying plates of drawings, in which:

FIG. 1 shows the distribution of mutations in rearranged VH3-23 gene segments deriving from XHIM patient and control donors.

In this figure, each histogram represents the percentage of VH3-23 sequences manifesting the number of mutations indicated in a given interval. Considering the remarkable mutation profile, patient C.Q. was considered separately from the other XHIM patients, whereas patient Z.A., whose mutation frequency was close to the baseline, was not included in this analysis. The number of V sequences analyzed in each group was: children n=33, control adults n=37, XHIM patients n=125, patient C.Q. n=28.

FIG. 2 shows a proposed scheme for the development of human B cells leading to an Ig gene hypermutation.

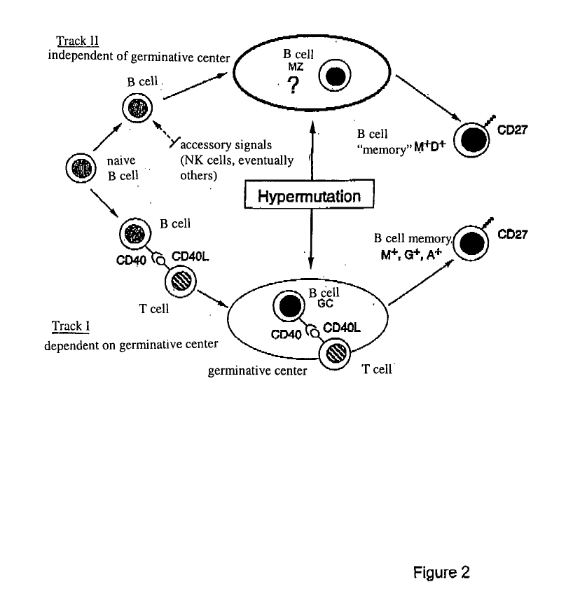

In this figure, track I corresponds to T-dependent responses occurring in germinative centers (CG), whereas track II is proposed as corresponding to T-independent responses, which may comprise the non-conventional assistance of T or NK cells. The splenic marginal zone (ZM), or equivalent sites in the Peyer plates or the lymphatic ganglia, might be the location of activation of B cells. Ig gene hypermutation takes place in each of these two tracks.

### DETAILED DESCRIPTION OF THE INVENTION

The theoretical principle underlying the present invention is Ig gene mutation of a B subpopulation defined in the absence of conventional T-B cooperation.

The B lymphocyte population, in man, comprises four sub-populations (U. Klein et al., J. Exp. Med. 188 [1998], 1679-1689):

The first consists of naive cells, still CD27 and IgM⁺IgD⁺ (M⁺D⁺27⁻, deriving from the bone marrow and representing about 60% of the B lymphocytes. They may be CD5⁺ (about 15%) or CD5⁻ (about 45%).

The other three consist of memory cells, representing the remaining 40%, all CD27⁺, with about 15% cells having performed the isotypical (M⁻D⁻27⁺) commutation and about 25% not having performed it (M⁺D⁺27⁺ about 15% and M⁺D⁻27⁺ about 10%).

It is interesting to note that this memory compartment, which thus represents about 40% of the B lymphocytes, represents only a few percent of them in mice. By contrast, man is capable of mutating all the B M⁺27⁻ cells, D⁺ and D⁻ alike, in the germinative centers.

First of all, it is known that the CD40 ligand (L) is the key molecule of the T-B interaction in T-dependent responses. Male human patients affected with the hyper IgM syndrome, with a mutilating mutation on CD40 L located on the X-chromosome, and the “knock-out” mice for this gene, do not form germinative centers, and in the absence of T-B cooperation, do not commutate the isotypes of the heavy chains, do not mutate their immunoglobulin genes, and have practically no IgG in circulation.

In the second place, the inventors of the present invention, by known techniques, have isolated the M⁺D⁺27⁺ population of such male human patients affected with the hyper IgM syndrome, and shown that the frequency of somatic mutations was more or less normal there, from patient to patient. Confronted with this very surprising result, they made the hypothesis that this M⁺D⁺27⁺ population, representing about 40% of the mutated cells in the normal adult, develops and diversifies in spite of the absence of germinative centers.

Again, the inventors of the present invention have also taken account of the fact that the M⁺D⁺27⁺ cells in man at birth represent about 1% of the B cells of the umbilical cord, and may exhibit a mutation rate of about 0.5 per sequence (variable), about 300 pb, whereas in subjects of adult age, the immunoglobulin genes have diversified and represent about 5% to 25% of the B compartments, with 5 to 10 mutations per sequence (variable).

Then it has been established, according to the present invention, that the development of this population parallels the appearance of responses to the T-independent antigens, whence the hypothesis that these cells may be responsible for the immune responses to bacteria having polysaccharide capsules—streptococci, pneumococci, meningococci, Haemophilus influenzae and other polysaccharide-encapsulated bacteria whose saccharide residues cannot be exhibited by CMH molecules—and that they may also be at the basis of the response to viruses of repetitive capsid structure, which trigger an essentially T-independent response, such as in particular the polio virus, influenza virus, encephalomyocarditis virus and others (M. F. Bachmann et al., “The influence of virus structure on antibody responses and virus serotype formation,” Immunol. Today 17[1996, No. 12], 553-558).

In fact, the inventors observed that those patients who showed a strong expansion of the B M⁺D⁺27⁺ cell population with a high rate of mutation are better protected than the others against the various types of pulmonary or ORL infections commonly observed in immunodeficient patients with IgG substitution.

Lastly, insofar as the response to bacterial antipolysaccharide vaccines does not manifest itself in splenectomized patients (D. Molrine et al., “Normal IgG and impaired IgM responses to polysaccharide vaccines in asplenic patients,” J. Inf. Dis. 179 [1999], 513-517), it may be supposed that the spleen, particularly the marginal zone, may be the place where these M⁺D⁺27⁺ cells are produced or stored.

Not to be bound to any particular theory, a plausible model of B cell development in a warm-blooded animal, including man, would seem to be one having two compartments corresponding to different types of exterior aggression to which the subject in question will be exposed during life. Man, among others, should thus have developed two B systems, one able to diversify in the germinative centers so as to respond with strong affinity to the thymodependent peptide antigens, and the other similar to that of the lymphoid tissues associated with the intestine (commonly termed GALT) and developed during ontogenesis in many species (in particular the rabbit, the sheep, the bovines), capable of diversifying independently of germinative centers and imparting protection against T-independent antigens in subjects where such cells are active.

To that end, account was taken of the results obtained by practicing the present invention, according to which Hyper IgM patients having a high M⁺D⁺27⁺ cell rate and bearing a diversified Ig receptor resist infections better than other patients not Hyper IgM but regularly receiving IgG substitution.

The experiments reported below will serve to illustrate aspects of the present invention and are not by any means to be regarded as limiting the same.

### Analysis by Separation and Cytometry of Flux of B IgM⁺IgD⁺CD27⁺ Cells

B M⁺D⁺27⁺ cells were screened by two-color staining on Ficoll-isopac purified cell suspensions enriched in B cells to 95-98% by magnetic cell separation using the MiniMACS (Miltenyi Biotech) system and one of the following reagents: 1) anti-human-FITC IgD (Caltag) and anti-human biotinyl CD27 (Ancell) plus Streptavidine-TriColor (Caltag); 2) anti-human-PE CD27 (Coulter-Immunotech), anti-IgD-FITC. One would prefer the latter combination for screening cells of XHIM patients, considering that the coloring of CD27⁺ populations present in a small percentage was sometimes reinforced with CD27-TriColor, according to our own observations.

The absence of IgD⁻CD27⁺ memory B cells was observed on Ficoll-purified PBMC by staining with anti-CD19-PC5 (Coulter-Immunotech), anti-IgD-FITC and anti-CD27-PE. A three-color analysis was performed on the B cells positive to CD19-PC5. Another characterization was performed on B cells enriched in CD19, stained with anti-IgD-FITC, IgM anti-human-PE (Caltag) and anti-CD27 biotinyl, followed by Streptavidine-TriColor. A three-color analysis was performed on cells positive to the CD27-TriColor barrier. Inasmuch as the IgD⁺CD27⁺ co-express IgM (data not shown), this population is called IgM⁺IgD⁺CD27⁺ (abbreviation M⁺D⁺27⁺).

### Analysis of Rearranged VH3-23 Gene Segment Sequences

From genomic DNA were extracted B D⁺27⁺ cells screened by digestion with K-proteinase. The rearranged VH3-23 gene segments were amplified from approximately 3000 cells via a Pfu Turbo polymerase (Statagene) using a semi-nested ACP strategy.

For the first round of amplification, a VH3-23 head fragment (5′-GGCTGAGCTGGCTTTTTCTTGTGG-3′) and a 3′J_{H} fragment mix (5′-TGAGGAGACGGTGACCAGGG-3′ and 5′-TGAGGAGACGGTGACCGTGG-3′ in proportions of 3:1) were used (45 seconds at 95° C., 60 seconds at 64° C., 90 seconds at 72° C. on 25 cycles). The second round of amplification was applied to the first reaction mixture using the same 3′J_{H} fragment mix and a VH3-23 intronic fragment (5′-GTGGAATGGATAAGAGTGA-3′) (45 seconds at 95° C., 60 seconds at 55° C. and 90 seconds at 72° C. on 25 cycles). The value of the background ACP error was determined under the same experimental conditions on B D⁺27⁻ cells derived from umbilical blood, with the same sample size (3000 cells). The ACP products purified on gel were cloned using the ACP products cloning kit Zero blunt TOPO PCR (Invitrogen). The sequences of colonies positive to VH3-23 were obtained by means of the cycle sequencing kit BigDye (Perkin-Elmer) and analyzed with an ABI310 genetic analyzer. The sequences obtained were compared with the VH3-23 gene of germinal lineate on 288 pairs of bases (pb) (from Glu1 to Cys92).

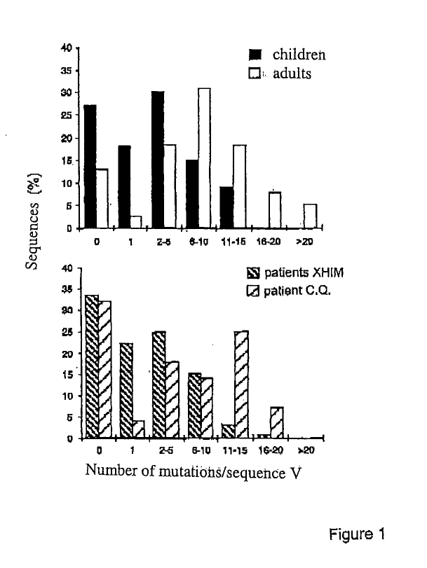

Using these materials and methods, somatic mutations were analyzed on rearranged VH3-23 sequences amplified from genomic DNA of B M⁺D⁺27⁺ cells. One patient (Z.A.) presented a mutation level near the baseline determined under the same experimental conditions on the M⁺D⁺27⁻ population; this patient had a peculiar medical history, having received and rejected a bone marrow graft three years before the present blood sampling. All the other patients, of whatever age, presented a mutation level similar to that observed in the control infants (0.5-1.7% for the total sequences and 0.9-1.9% for the mutated sequences, with 0-15 mutations per V sequence), except one who, remarkably enough given early age (C.Q., 5 years), presented a mutation frequency close to that of a control adult (2.2% for total sequences, 3.27% for mutated sequences, 0-18 mutations per V sequence) (see Table I and FIG. 1). Analysis of all the sequences revealed a normal distribution of mutations with a grouping or aggregation and a selection for the CDR replacement mutations. In all of the patients, most of the sequences exhibited different V_{H}-D-J_{H} junctions, indicating the absence of any specific VH3-23 clonal expansion.+

TABLE I
Somatic mutations in rearranged VH3-23 genes in B cells of peripheral blood,
IgD⁺IgM⁺CD27⁺, of XHIM patients

| | | | | | | Mutations | | | |
|---|---|---|---|---|---|---|---|---|---|
| | | | | | | | | Frequency/ | |
| | | | | | | | | sequences | |
| | | Age | %B D⁺M⁺27⁺ | No. of Sequences | | | | Total | Mutated |
| | Donor | (yrs) | Cells | Total | Mutated | Range | No. | (%) | (%) |
| XHIM | C.Q. | 5 | 1 | 28 | 19(67%) | 0-18 | 178 | 2.2 | 3.27 |
| patients | C.R. | 7 | 1.5 | 18 | 10(89%) | 0-19 | 80 | 1.7 | 1.9 |
| | L.P. | 7 | 2 | 19 | 13(68%) | 0-12 | 61 | 1.1 | 1.82 |
| | A.N. | 7 | 2 | 18 | 9(50%) | 0-7 | 27 | 0.52 | 1 |
| | L.Ch. | 8 | 1 | 27 | 16(60%) | 0-10 | 41 | 0.52 | 0.89 |
| | Z.A. | 15 | 2 | 24 | 12(50%) | 0-1 | 12 | 0.17 | 0.34 |
| | B.M₂ | 16 | 4 | 23 | 10(43%) | 0-15 | 40 | 0.6 | 1.33 |
| | F.F₁ | 21 | 60 | 20 | 19(95%) | 0-9 | 75 | 1.3 | 1.37 |
| Healthy | D1 | 4 | 7 | 15 | 8(54%) | 0-14 | 45 | 1 | 1.95 |
| controls | D2 | 5 | 7 | 19 | 17(89%) | 0-13 | 78 | 1.5 | 1.7 |
| | D3 | 16 | 7 | 14 | 18(93%) | 0-19 | 190 | 3.22 | 3.47 |
| | D4 | adult | 10 | 23 | 19(83%) | 0-22 | 182 | 2.75 | 3.84 |
| Blood from | C1 | | 1 | 17 | 11(64%) | 0-2 | 12 | 0.24 | 0.37 |
| umbilical | C2 | | 1 | 25 | 5(20%) | 0-2 | 6 | 0.08 | 0.4 |
| | C3 | | 1 | 16 | 4(25%) | 0-1 | 4 | 0.08 | 0.34 |
| Baseline | | | | 44 | 12(27%) | 0-2 | 14 | 0.1 | |
| (B D⁺27⁺ cells) | | | | | | | | | |

The variable proportion of non-mutated sequences obtained in the M⁺D⁺27⁺ population (5% to 60%) may correspond to the variable purity of the screened population when present at a low frequency. This proportion was reduced considerably when the M⁻D⁺27⁺ cells were present in large number (for example, patient F.F. with 60% B M⁺D⁺27⁺ cells had 95% mutated V sequences; see Table I). According to three control samples of umbilical cord blood, the frequency of mutations in the M⁺D⁺27⁺ population was close to the baseline in two cases and slightly above in one case (double the base level).

It was thus shown that this particular population of B M⁺D⁺27⁺ cells has a path of differentiation proper to itself. It presents some receptors unique to itself, which in turn can be used specifically. Those skilled in the art are able to select on the basis of the information contained here and their own knowledge, if necessary performing iterative tests, surface markers proper to this subpopulation of B cells, the mobilization and/or stimulation of which according to the invention serves to provide a treatment beneficial to the patients concerned.

By way of indication but not of limitation, such markers may be CD21 and IgD associated with CD27. It has also been found that another marker, CD1c, strongly expressed on these cells, can serve for vaccinal stimulation.

Given the critical observation according to which this population mutates its Ig genes in independent manner, the vaccination means employed in the method of immunization according to the invention are able considerably to improve the antibodies produced, inducing specific proliferation of the clones concerned and thus augmenting the rate of mutation.

A vaccine according to the invention may comprise an immunogenic composition containing a conjugate. According to one embodiment of said vaccine, the agent capable of provoking a T-independent immune response may be fixed by covalency to liposomes.

The immunogenic composition thus employed may be combined with a pharmaceutically acceptable support in a pharmaceutical composition.

Furthermore, diagnostic means based on the present invention permit identification and/or quantification of this population of particular B cells present in the blood, serving to test/diagnose the efficacy of a vaccination. In practice, to do this, VH genes specifically involved in this response are analyzed, both on the level of their mutation rate and on the level of expansion of the clone involved.

To perform such a diagnosis, one may work on samples of about 10 ml uncoagulated blood, from which the leucocytes are extracted, while bactericidal tests or controls are performed by methods known to those skilled in the art.

Further, given the rôle of this population of specific B cells in the antibacterial and/or antiviral responses here under consideration, an abnormal expansion of this population of B cells is observed in certain auto-immune manifestations. Here again, the quantification of this population of blood cells serves to diagnose the presence and/or size of these abnormal populations in a very simple manner.

Instead, this time, of inducing the proliferation of this subpopulation of B cells considered in the context of a vaccination, one may likewise, by means of inhibitor molecules specific to this population, inhibit the proliferation of these cells in such pathology. Those skilled in the art are able to find and test, for the purpose of selection, such specific inhibitor molecules in the subpopulation of B M⁺D⁺27⁺ cells. In this connection, it should be noted also that a certain proportion of B lymphomes emerge in the course of these auto-immune syndromes, and that the phenotype of these lymphomes has often been found to correspond to that of the B M⁺D⁺27⁺ cells. Thus, the invention also contemplates means of controlling the growth of these cells both in the period of hyperplasia and in the tumoral period, these means being based on specific inhibitors of the subpopulation of B M⁺D⁺27⁺ cells.

Consequently, another object of the invention is a composition to inhibit or negatively control an auto-immune response to the bacterial polysaccharide antigens and to the protein structures of virus capsids, comprising an efficacious quantity of specific inhibitor molecules for the subpopulation of B M⁺D⁺27⁺ cells.

For either the stimulation or mobilization or the inhibition of the said specific B cells, according to the invention, one may for example proceed by intradermal injection of bacterial polysaccharide, meningococcic, pneumococci, etc., with molecules capable of specifically stimulating the said M⁺D⁺27⁺ population.

In practice, and solely by way of example, a dose of immunogenic composition from about 0.01 μg to about 10 μg per kilogram body weight of the individual treated is appropriate.

The support of the immunogenic composition may be of any kind, in particular a saline solution, Ringer's solution, or a phosphate-buffered saline solution. In practice, the immunogenic composition will advantageously comprise an adjuvant.

The said immunogenic composition may comprise an immunogenic conjugate, and it may be administered to an individual, for example, in an immunogenic agent dose from about 0.01 μg to about 10 μg per kilogram body weight.

Even in human patients aged 2 to 60 years, the injectable compositions prepared according to the invention serve for efficacious reinforcement, according to the specific receptors used, of either the stimulation or mobilization or the inhibition of B M⁺D⁺27⁺ cells inducing an immune response in the patient treated.

The originality of the concept according to the invention consists in the insight that the B cells responsible for the antibacterial and partially antiviral response as set forth above are in fact already present at the earliest age (under 1 year in children) with well-diversified receptors, and that with the aid of specific markers for these populations, one can stimulate them and thus protect very young children with uncoupled polysaccharide antigens, which was not the case heretofore.

Incidentally, there is no reason to suspect any particular toxicity on the part of the products and means according to the present invention.

In conclusion, it has been shown according to the invention that it is possible to induce or reinforce a T-independent antibacterial immune response in warm-blooded animals, including man, and more specifically in young children under 2 years of age and in persons over 65 years of age, by administering a suitable quantity of vaccine capable of mobilizing the B M⁺D⁺27⁺ cells specifically. Administration of the immunization means according to the invention may be performed by injection via conventional routes, specifically but not exclusively by the intravenous, intraperitoneal, intradermal or intramuscular route, as well as by other conventional routes of administration, provided the vectors or supports and the adjuvants used are adapted from case to case by those skilled in the art, who to do so will call upon their own knowledge, and may be prompted to perform tests in order to identify a mode of administration and quantities to be recommended.

The invention will also serve, if desired, for specific inhibition of the action of the said B M⁺D⁺27⁺ cells.

In vitro, this same subpopulation of B cells may be used to diagnose a state of infection by polysaccharide antigens, and likewise to test the efficacy of a vaccination as above mentioned on a blood sample from an immunodeficient subject treated according to the invention, by comparison of the test results, for example by an ELISA methodology, with blood samples from subjects manifesting a natural immune response to the same polysaccharide antigens and tested in parallel.

1. Immunogenic composition able to provide an immune response to polysaccharide antigens and/or protein structures of virus capsids, characterized by comprising, in a pharmaceutically acceptable support, a therapeutically efficacious quantity of at least one specific surface marker for B cells of the M⁺D⁺27⁺ population, including those cells of such a subpopulation of B cells which are not in a germinative center.

2. Immunogenic composition according to claim 1, in which the support is chosen in the group consisting of a saline solution, Ringer's solution, and a phosphate-buffered saline solution.

3. Immunogenic composition according to either of claims 1 and 2, in which the immunogenic composition also comprises an adjuvant.

4. Immunogenic composition according to any of claims 1 to 3, characterized by comprising an immunogenic conjugate.

5. Immunogenic composition according to claim 1, characterized by comprising specific surface markers for the B cells of subpopulation M⁺D⁺27⁺, in particular markers chosen among CD21, IgD associated with CD27, and CD1c.

6. Vaccine for obtaining protection against T-independent antibacterial infections, comprising a quantity of the immunogenic composition according to any of claims 1 to 5, capable of provoking a T-independent immune response.

7. Vaccine according to claim 6, in which the said immunogenic composition comprises a conjugate.

8. Vaccine according to claim 6, in which the agent capable of provoking a T-independent immune response is fixed to liposomes by covalency.

9. Use of the immunogenic composition according to any of claims 1 to 5 for preparation of a medication for obtaining immune responses in warm-blooded animals, including man, to bacterial polysaccharide antigens and to protein structures of virus capsids.

10. Use according to claim 9, in which the said immunogenic composition is administered to an individual in an immunogenic dose from about 0.01 μg to about 10 μg per kilogram body weight.

11. Use of the vaccine according to any of claims 6 to 8 for preparation of a medication to obtain immune responses in warm-blooded animals, including man, to bacterial polysaccharide antigens and to the protein structures of virus capsids.

12. Use of the vaccine according to any of claims 6 to 8 for preparation of a medication for protection against an infection by streptococci, pneumococci, meningococci or Haemophilus influenzae.

13. Pharmaceutical composition comprising an immunogenic composition according to any of claims 1 to 5 and a pharmaceutically acceptable support.

14. Process of diagnosis in vitro, either of states requiring vaccination, or of the efficacy of a vaccination against a state of infection by polysaccharide antigens, characterized by the detection and/or quantification of B cells of subpopulation M⁺D⁺27⁺ in a blood sample.

15. Process of diagnosis in vitro according to claim 14, characterized by comprising analysis of the VH genes specifically involved in the said immune response, advantageously both at the level of their mutation rate and at the level of expansion of the clone involved.

16. Process of diagnosis in vitro according to either of claims 14 or 15, characterized by employment thereof on a blood sample collected from an immunodeficient subject and comparison of the results of the test with those of blood samples collected from subjects manifesting a natural immune response to the same polysaccharide antigens.

17. Process of diagnosis in vitro according to claim 16, characterized by use of a blood sample of about 10 ml.

18. Use of the immunogenic composition according to any of claims 1 to 5 for preparation of a medication having an inhibitive action on the auto-immune response in a subject by specific inhibition of the action of B M⁺D⁺27⁺ cells.
